(19) United States
(12) Patent Application Publication
(10) Pub. No.: US 2015/0148577 A1
(43) Pub. Date: May 28, 2015

(54) SEPARATION OF ISO-OLEFINS FROM PARAFFINS IN THE C19 TO C22 RANGE

(71) Applicant: UOP LLC, Des Plaines, IL (US)

(72) Inventors: Santi Kulprathipanja, Inverness, IL (US); Stephen W. Sohn, Arlington Heights, IL (US)

(73) Assignee: UOP LLC, Des Plaines, IL (US)

(21) Appl. No.: 14/089,996

(22) Filed: Nov. 26, 2013

(51) Int. Cl. C07C 7/12; C07C 2/64

(52) U.S. Cl. CPC C07C 7/12; C07C 2/64
USPC 585/448; 585/829

(57) ABSTRACT

A process is presented for the separation of iso-olefins from a hydrocarbon mixture comprising paraffins and olefins. The process includes an adsorption separation system, wherein the adsorbent is selected according to the properties of the material that is desired to be adsorbed. The process also includes a selection of a desorbent, which can comprise a mixture, to provide for an enhanced recovery of the adsorbed material and a separation of the iso-olefins from paraffins.

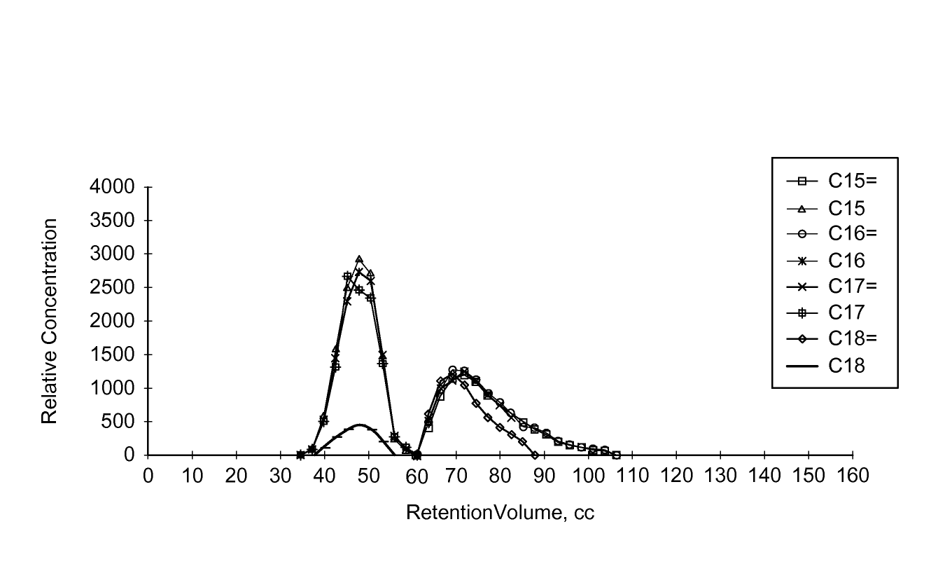

### FIELD OF THE INVENTION

The present invention relates to the field of benzene alkylation. In particular, this invention relates to the production of the precursor olefinic materials used in the production of surfactants.

### BACKGROUND

Alkylation of benzene produces alkylbenzenes that may find various commercial uses, e.g., alkylbenzenes can be sulfonated to produce surfactants, for use in detergents. In the alkylation process, benzene is reacted with an olefin the desired length to produce the sought alkylbenzene. The alkylation conditions comprise the presence of homogeneous or heterogeneous alkylation catalyst such as aluminum chloride, hydrogen fluoride, or zeolitic catalysts and elevated temperature.

Various processes have been proposed to alkylate benzene. One commercial process involves the use of hydrogen fluoride as the alkylation catalyst. The use and handling of hydrogen fluoride does provide operational concerns due to its toxicity, corrosiveness and waste disposal needs. Solid catalytic processes have been developed that obviate the need to use hydrogen fluoride. Improvements in these solid catalytic processes are sought to further enhance their attractiveness through reducing energy costs and improving selectivity of conversion while still providing an alkylbenzene of a quality acceptable for downstream use such as sulfonation to make surfactants.

Alkylbenzenes, to be desirable for making sulfonated surfactants must be capable of providing a sulfonated product of suitable clarity, biodegradability and efficacy. With respect to efficacy, alkylbenzenes having higher 2-phenyl contents are desired as they tend, when sulfonated, to provide surfactants having better solubility and detergency. Thus, alkylbenzenes having a 2-phenyl isomer content in the range from about 30 to about 40 percent are particularly desired.

The production of alkylbenzenes requires the production of olefins for alkylation of the benzene. The olefins for detergents are preferably linear alpha olefins, which produce alkylbenzenes having favorable biodegradability properties. The recovery of olefins from a hydrocarbon stream affects the ability to economically produce the alkylbenzenes. Improvements in the production and recovery of olefins can improve the economics of alkylbenzene production.

### SUMMARY

The present invention is a process that utilizes the properties of the adsorbent and desorbent to improve the separation of components from a mixture.

A first embodiment of the invention is a process for separating olefins from a hydrocarbon stream comprising passing a hydrocarbon stream comprising C19 to C22 olefins and paraffins to an adsorption separation system, thereby creating an extract stream comprising iso-olefins and normal olefins, and a raffinate stream having a reduced iso-olefin and normal olefin content; wherein the adsorbent comprises a alkali substituted zeolite, and wherein the adsorption separation system uses a desorbent that comprises a naphthene or naphthene mixture. An embodiment of the invention is one, any or all of prior embodiments in this paragraph up through the first embodiment in this paragraph wherein the alkali substituted zeolite is sodium substituted X zeolite. An embodiment of the invention is one, any or all of prior embodiments in this paragraph up through the first embodiment in this paragraph wherein the desorbent comprises a naphthene in the C5 to C10 range. An embodiment of the invention is one, any or all of prior embodiments in this paragraph up through the first embodiment in this paragraph wherein the desorbent comprises a naphthene selected from the group cyclohexane, methylcyclohexane, methylcyclopentane, cyclopentane and mixtures thereof An embodiment of the invention is one, any or all of prior embodiments in this paragraph up through the first embodiment in this paragraph wherein the iso-olefins comprise mono-methyl, mono-ethyl and mono-propyl olefins. An embodiment of the invention is one, any or all of prior embodiments in this paragraph up through the first embodiment in this paragraph further comprising passing the extract stream to a first separation unit to generate an extract process stream and an extract desorbent stream. An embodiment of the invention is one, any or all of prior embodiments in this paragraph up through the first embodiment in this paragraph further comprising passing the extract desorbent stream back to the adsorption separation column. An embodiment of the invention is one, any or all of prior embodiments in this paragraph up through the first embodiment in this paragraph further comprising passing the raffinate stream to a second separation unit to generate a raffinate process stream and a raffinate desorbent stream. An embodiment of the invention is one, any or all of prior embodiments in this paragraph up through the first embodiment in this paragraph further comprising passing the raffinate desorbent stream back to the adsorption separation column. An embodiment of the invention is one, any or all of prior embodiments in this paragraph up through the first embodiment in this paragraph wherein the hydrocarbon stream is generated from gas to liquids technology to generate a feedstream comprising paraffins. An embodiment of the invention is one, any or all of prior embodiments in this paragraph up through the first embodiment in this paragraph further comprising passing the feedstream comprising paraffins to a paraffin to olefin conversion zone to generate the hydrocarbon stream comprising C19 to C22 olefins and paraffins.

A second embodiment of the invention is a process for the production of long chained alkylbenzenes comprising passing a hydrocarbon stream comprising C19 to C22 olefins and paraffins through an adsorption separation system, thereby generating an extract stream comprising C19 to C22 iso-olefins and a raffinate stream comprising paraffins, wherein the adsorbent in the adsorption separation system is a sodium based zeolite; passing the extract stream and a benzene stream to an alkylation reactor to generate an alkylbenzene process stream. An embodiment of the invention is one, any or all of prior embodiments in this paragraph up through the second embodiment in this paragraph wherein the adsorption separation system uses a desorbent comprising a naphthene. An embodiment of the invention is one, any or all of prior embodiments in this paragraph up through the second embodiment in this paragraph wherein the desorbent comprises a naphthene in the C5 to C10 range. An embodiment of the invention is one, any or all of prior embodiments in this paragraph up through the second embodiment in this paragraph wherein the desorbent is selected from the group cyclohexane, methylcyclohexane, methylcyclopentane, cyclopentane and mixtures thereof. An embodiment of the invention is one, any or all of prior embodiments in this paragraph up through the second embodiment in this paragraph further comprising passing the extract stream to a first separation unit to generate an extract process stream and an extract desorbent stream, and then passing the extract process stream to the alkylation reactor.

A third embodiment of the invention is a process for separating olefins from a hydrocarbon stream, comprising passing a feedstream to an adsorption separation system to selectively adsorb olefins, and to generate a raffinate stream having a reduced olefin content; and passing a desorbent to the adsorption separation system to displace the selectively adsorbed olefins to generate an extract stream comprising olefins; wherein the adsorbent in the adsorption separation system is a sodium substituted X zeolite, and wherein the desorbent is a naphthene. An embodiment of the invention is one, any or all of prior embodiments in this paragraph up through the third embodiment in this paragraph wherein the desorbent is selected from the group consisting of cyclohexane, methylcyclohexane, methylcyclopentane, cyclopentane and mixtures thereof. An embodiment of the invention is one, any or all of prior embodiments in this paragraph up through the third embodiment in this paragraph wherein the feedstream comprises paraffins and olefins in the C19 to C22 range. An embodiment of the invention is one, any or all of prior embodiments in this paragraph up through the third embodiment in this paragraph further comprising passing the extract stream to a fractionation unit to generate an extract product stream and a desorbent stream.

Other objects, advantages and applications of the present invention will become apparent to those skilled in the art from the following detailed description and drawing.

### BRIEF DESCRIPTION OF THE DRAWING

FIGURE is plot of a pulse test run of a synthetic feed for testing the adsorbent and desorbent.

### DETAILED DESCRIPTION

Heavier hydrocarbons beyond the detergent range hydrocarbons are being used for surfactants in new environments. One such area is the enhanced oil recovery (EOR) business, where surfactants are injected into oil fields to change the properties of the oil in the field and to subsequently increase the flow of oil. For enhanced oil recovery, biodegradability is an unimportant property, and therefore, less biodegradable surfactants can be used, while seeking to obtain more optimal surfactants that enhance the flow of oil from an oil field. For extremely heavy oil, heavier detergent compounds can be employed, but the components, that is, the normal paraffins need to be separated and recovered. The separation of paraffins using absorption-separation technology can be limited to a relatively narrow range of carbon numbers for paraffins, such as a spread of about 4 to 5 carbons to be separated. In this present process, the aim is to recover normal paraffins in the C19 to C22 range.

Surfactants, and detergents, utilize olefins for the manufacture of precursors, which are subsequently sulfonated to generate the surfactants. An effective detergent is designed to comprise an aromatic group and a long chained alkyl group. The detergent is preferentially made with a linear alkylbenzene (LAB) constituent. The LAB is preferentially formed alkylating benzene with a linear alpha-olefin, which in turn forms a 2-alkylbenzene product.

Surfactants are also useful in other fields. One such area is the field of enhanced oil recovery (EOR). Enhanced oil recovery is also looking at different requirements, and different limitations from those associated with normal detergents. Differences include a lack of need for environmental breakdown of the surfactants, and surfactants having a larger non-polar component. The larger non-polar component can be reflected in a structure having a larger tail, or a longer carbon chain. The EOR formulation will select a hydrophobic chain length to match the characteristics of the oil in the oil reservoir. Heavier oils will require longer chain molecules to facilitate the oil displacement.

A hydrocarbon stream having olefins can be generated in several ways. The separation of hydrocarbons having similar molecular weights, and similar structures, such as paraffins and olefin analogs of those paraffins can be separated through an adsorption separation system. Adsorption separation systems are known to those skilled in the art, and descriptions can be found in many sources of literature. One early patent covering adsorption separation systems is U.S. Pat. No. 2,985,589 to Broughton, et al. issued in 1961. The adsorption separation process is a simulated moving bed process of solid adsorbent and counter-current extraction, where the feed, desorbent, raffinate and extract streams are moved sequentially along the adsorption separation column between adjacent adsorption chamber beds. The system will have typically between 4 and 24 chamber beds, but can have more if the separation process for a particular system so requires. While the mechanical aspects of the process is known, such as controlling flows and shifting feeds, the separation of a constituent from a particular mixture can be difficult, and involves many considerations.

These consideration, beyond the mechanical aspect of operating the separation process, include selecting an appropriate adsorbent and an appropriate desorbent.

The choice of adsorbent and the choice of desorbent can have a significant impact on the selectivity of components separated and the quality of the extract stream. A problem that can occur with a poor desorbent selection can be with a strong desorbent. With a strong desorbent, the adsorbent capacity is reduced, and the separation and recovery of the adsorbed material is reduced. In the separation of olefins from an olefin-paraffin mixture, this results in a reduced olefin recovery.

The present invention has found for a separation of olefins from a feedstream comprising olefins and paraffins in the C19 to C22 range, one needs a particular adsorbent, and a particular desorbent. Problems that can occur, include non-uniform recovery of the adsorbed material. Different desorbents will displace the different adsorbed constituents at different rates. For example C19 to C22 olefins may be uniformly adsorbed, but a desorbent might preferentially displace C19 olefins over the heavier olefins. The separation requires an appropriate matching of adsorbent for selective adsorption of a particular molecule, or range of molecules, and the appropriate matching of a desorbent for the desorption of all the adsorbed molecules in the desired range. Preferably, the desorbent provides for a more uniform desorption of all the adsorbed molecules.

One current weakness in an adsorption separation system is the strength of the desorbent for one end of an adsorbed hydrocarbon range. This can be particularly true for a strong desorbent, where the desorbent will preferentially desorb material from one end of a hydrocarbon range. With a strong desorbent, the adsorbent capacity for olefins will be reduced at one end of the range, and the recovery of olefins and paraffins in the desired range will be reduced, and even skewed.

The present process provides for the separation of olefins from a hydrocarbon stream, wherein the stream comprises olefins and paraffins in the C19 to C22 range. The hydrocarbon stream is passed to an adsorption separation system, to generate an extract stream comprising iso-olefins and normal olefins, and a raffinate stream having a reduced olefin content. The adsorbent comprises an alkali substituted zeolite, and the adorption-separation uses a desorbent comprising smaller hydrocarbons. Alkali elements useable for substitution include lithium, sodium, potassium, rubidium and cesium. A mixture of alkali substituted zeolites can also be used. A preferred alkali element is sodium, and the preferred adsorbent is NaX. One preferred desorbent comprises a naphthene, or a naphthene mixture. In a preferred embodiment, the adsorbent comprises an alkali substituted zeolite, and a preferred zeolite is sodium substituted X zeolite. The X zeolite can include a binder.

In one embodiment, the adsorbent comprises a binderless adsorbent comprising an

X zeolite, wherein the binder in the zeolite has been converted to zeolite X. The adsorbent can include a water content from about 2.5 to 5 wt % of the binderless adsorbent. In another embodiment, the adsorbent comprises an X zeolite with a silica to alumina molar ratio between 2 and 2.6, with a preferred molar ratio between 2.4 and 2.6. In one embodiment, the adsorbent comprises adsorbent particles having an average crystalline size between 1 and 3 μm. The adsorbent can include preferred ratios of alkali substituted components on the zeolite. The adsorbent can also be treated to activate the adsorbent. The activation conditions can include heating the adsorbent to a temperature between 500° C. and 700° C.

The adsorption process is carried out at a temperature between 100° C. and 180° C., with a preferred temperature between 120° C. and 160° C.

A preferred desorbent comprises a naphthene in the range from C5 to C10 carbon atoms. A mixture of naphthenes in this range can include cyclohexane, methylcyclohexane, methylcyclopentane, and cyclopentane. The separation of olefins includes the separation of iso-olefins from the hydrocarbon stream, and includes mono-methyl, mono-ethyl and mono-propyl olefins. The hydrocarbon stream comprising paraffins and olefins is generated by a process in gas to liquids technology, wherein the paraffins and olefins are generated from smaller hydrocarbon molecules through an oligomerization process or a dimerization process. The gas to liquids technology can start with a feed stream that has originated from a natural gas source, or from a syn-gas source. For processes where the conversion of compounds to larger hydrocarbons includes the conversion of oxygenates, or the generation of oxygenates, such as in a syn-gas process, the process includes a hydrotreatment step, or an extraction step to remove all the oxygenates. The process may further include a dehydrogenation step if the process generates paraffins. The conversion process for gas to liquids converts smaller hydrocarbon molecules to a hydrocarbon stream comprising paraffins and olefins in the C19 to C22 range.

The process can further include passing the extract stream to a first separation unit to generate an extract process stream and an extract desorbent stream. The first separation unit can comprise a fractionation column, with the extract process stream is a bottoms stream comprising the iso-olefins, and the extract desorbent stream is an overhead stream comprising the desorbent. The extract desorbent stream is passed back to the adsorption separation system for reuse.

The process can further include passing the raffinate stream to a second separation unit to generate a raffinate process stream and a raffinate desorbent stream. The second separation unit can comprise a fractionation column, with the raffinate process stream is a bottoms stream comprising the iso-olefins, and the raffinate desorbent stream is an overhead stream comprising the desorbent. The raffinate desorbent stream is passed back to the adsorption separation system for reuse. The recovered raffinate can be passed to a dehydrogenation step for the generation of more olefins.

For the separation of heavy paraffins and olefins from the desorbent, an additional constraint exists. The current recovery of the normal paraffins, or olefins, in lower carbon ranges from the extract, or raffinate, mixture containing the desorbent and the separated components is to fractionate the mixture. The desorbent is recovered in the fractionation column overhead, while the separated components are recovered from column bottoms. With higher molecular weight hydrocarbons in order to separate the high molecular weight hydrocarbons from the desorbent, sufficient heat must be applied to boil the heavier hydrocarbons. However, at those temperatures the heavier hydrocarbons will crack, and will render the separation process ineffective. If the fractionation is performed under a vacuum, in order to reduce the temperature of operation, then the desorbent can not be condensed in a manner that does not require expensive technology, such as a significant refrigeration system.

For a significant recovery, the process can include adding a diluents to the extract and raffinate streams. The diluent is a hydrocarbon mixture having an intermediate range of carbon numbers between the desorbent and the carbon numbers of the extract and raffinate streams. The use of an intermediate range provides for a sufficient vapor to separate the desorbent from the mixture of extract, desorbent and diluent. The fractionation system utilizes two fractionation columns for the extract stream recovery and two fractionation columns for the raffinate stream recovery. In each pair of columns, the desorbent is recovered as an overhead stream from the first column, and the second column then separates the diluent from either the extract or the raffinate heavy hydrocarbons. The diluents is passed out as an overhead stream from the second column and recycled, and the extract, or raffinate are passed out as bottoms from the second column.

The normal operation of a fractionation column can be at atmospheric pressure or higher, but where the temperatures required to perform the fractionation are too great, the pressure can be reduced to below atmospheric. The operation of the first and second separation columns are operated at or above atmospheric conditions. These conditions allow for the condensation of the desorbent during the separation process. The operating conditions include temperatures between 35° C. and 260° C., and pressures between 100 kPa and 500 kPa. Considerations include the ability to condense the desorbent to create a reflux stream, and to boil the other components to create a vapor stream flowing upward from the bottom of the columns.

While the invention has been described with what are presently considered the preferred embodiments, it is to be understood that the invention is not limited to the disclosed embodiments, but it is intended to cover various modifications and equivalent arrangements included within the scope of the appended claims.

What is claimed is:

1. A process for separating olefins from a hydrocarbon stream comprising:
passing a hydrocarbon stream comprising C19 to C22 olefins and paraffins to an adsorption separation system, thereby creating an extract stream comprising iso-olefins and normal olefins, and a raffinate stream having a reduced iso-olefin and normal olefin content;
wherein the adsorbent comprises an alkali substituted zeolite, and wherein the adsorption separation system uses a desorbent that comprises a naphthene or naphthene mixture.

2. The process of claim 1 wherein the alkali substituted zeolite is sodium substituted X zeolite.

3. The process of claim 1 wherein the desorbent comprises a naphthene in the C5 to C10 range.

4. The process of claim 3 wherein the desorbent comprises a naphthene selected from the group cyclohexane, methylcyclohexane, methylcyclopentane, cyclopentane and mixtures thereof

5. The process of claim 1 wherein the iso-olefins comprise mono-methyl, mono-ethyl and mono-propyl olefins.

6. The process of claim 1 further comprising passing the extract stream to a first separation unit to generate an extract process stream and an extract desorbent stream.

7. The process of claim 6 further comprising passing the extract desorbent stream back to the adsorption separation column.

8. The process of claim 1 further comprising passing the raffinate stream to a second separation unit to generate a raffinate process stream and a raffinate desorbent stream.

9. The process of claim 8 further comprising passing the raffinate desorbent stream back to the adsorption separation column.

10. The process of claim 1 wherein the hydrocarbon stream is generated from gas to liquids technology to generate a feedstream comprising paraffins.

11. The process of claim 10 further comprising passing the feedstream comprising paraffins to a paraffin to olefin conversion zone to generate the hydrocarbon stream comprising C19 to C22 olefins and paraffins.

12. A process for the production of long chained alkylbenzenes comprising:
passing a hydrocarbon stream comprising C19 to C22 olefins and paraffins through an adsorption separation system, thereby generating an extract stream comprising C19 to C22 iso-olefins and a raffinate stream comprising paraffins, wherein the adsorbent in the adsorption separation system is a sodium based zeolite;
passing the extract stream and a benzene stream to an alkylation reactor to generate an alkylbenzene process stream.

13. The process of claim 12 wherein the adsorption separation system uses a desorbent comprising a naphthene.

14. The process of claim 13 wherein the desorbent comprises a naphthene in the C5 to C10 range.

15. The process of claim 14 wherein the desorbent is selected from the group cyclohexane, methyl cyclohexane, methyl cyclopentane, cyclopentate and mixtures thereof

16. The process of claim 12 further comprising passing the extract stream to a first separation unit to generate an extract process stream and an extract desorbent stream, and then passing the extract process stream to the alkylation reactor.

17. A process for separating olefins from a hydrocarbon stream, comprising:
passing a feedstream to an adsorption separation system to selectively adsorb olefins, and to generate a raffinate stream having a reduced olefin content; and
passing a desorbent to the adsorption separation system to displace the selectively adsorbed olefins to generate an extract stream comprising olefins;
wherein the adsorbent in the adsorption separation system is a sodium substituted X zeolite, and wherein the desorbent is a naphthene.

18. The process of claim 17 wherein the desorbent is selected from the group consisting of cyclohexane, methylcyclohexane, methylcyclopentane, cyclopentane and mixtures thereof

19. The process of claim 17 wherein the feedstream comprises paraffins and olefins in the C19 to C22 range.

20. The process of claim 17 further comprising passing the extract stream to a fractionation unit to generate an extract product stream and a desorbent stream.
